(19) United States
(12) Patent Application Publication
(10) Pub. No.: US 2006/0149129 A1
(43) Pub. Date: Jul. 6, 2006

(54) Catheter with multiple visual elements

(76) Inventors: H. David Watts, Mill Valley, CA (US); John Higgins, Los Altos, CA (US); Fred R. Seddiqui, Los Altos, CA (US); Alex C. Neil, Daly City, CA (US); Rupesh Desai, San Jose, CA (US)

Correspondence Address: SQUIRE, SANDERS & DEMPSEY L.L.P., 1 MARITIME PLAZA, SUITE 300, SAN FRANCISCO, CA, 94111, US

(21) Appl. No.: 11/215,660

(22) Filed: Aug. 29, 2005

Related U.S. Application Data
(63) Continuation-in-part of application No. 11/030,559, filed on Jan. 5, 2005.

(51) Int. Cl. A61B 1/00; A61B 1/06

(52) U.S. Cl. 600/113; 600/173; 600/179

(57) ABSTRACT

A catheter assembly is provided with a first visual device and a second visual device. The second visual device is movable with respect to the first visual device to provide image feedback of a lesion from a different prospective than the first visual device. The second visual device can be moved before, contemporaneously with, or subsequent to a diagnostic or therapeutic procedure. The second visual device can include an elongated tube that can be extended into or retracted out from an insertion tube of the catheter assembly.

### CROSS REFERENCE

This application is a continuation-in-part of application Ser. No. 11/030,559, filed Jan. 5, 2005.

### TECHNICAL FIELD

The present invention relates to implantable or insertable medical devices for capturing images. More specifically, the present invention relates to a catheter system which includes a fixed visual element and a movable visual element for allowing a user to receive multiple images of a lesion at different angles.

### BACKGROUND

An endoscope is a medical device consisting of a camera mounted on a flexible tube. Small instruments can be used to take samples of suspicious tissues or to perform other surgical procedures through the endoscope. In gastrointestinal endoscopy, this device is inserted through the mouth or anus. For other areas, small incisions are made. There are many types of endoscope, and they are named in relation to the organs or areas they explore. Endoscopes used to look directly at the ovaries, appendix, or other abdominal organs, for example, are called laparoscopes (laparoscopy). Other endoscopes are inserted through incisions to look at joints (arthroscopy). Still others are used to view the inside of the bladder (cystoscopy) or the lungs (bronchoscopy). Laparoscopy is usually performed under general anesthesia, while most other endoscopies can be performed with the patient sedated. With appropriate sedation, the patient should experience little if any discomfort. An endoscopy may be performed for a variety of signs and symptoms, including bleeding, pain, difficulty swallowing, and a change in bowel habits. Exams of the colon may also be performed to screen for colon polyps and colon cancer.

An anoscopy is a procedure that enables a physician to view the anus, anal canal, and lower rectum using a speculum. First, the health care provider performs a digital rectal exam by inserting a lubricated, gloved finger into the rectum to determine if anything will block the insertion of the scope. He or she then inserts a lubricated metal or plastic anoscope a few inches into the rectum. This enlarges the rectum to allow the health care provider to view the entire anal canal using a light. A specimen for biopsy can be taken if needed. As the scope is slowly removed, the lining of the anal canal is carefully inspected. This test may be used to determine whether the patient has hemorrhoids, anal polyps, tumors, inflammation, fissures, or infection.

Sigmoidoscopy is the internal examination of the rectum, sigmoid colon, and distal large bowel using a “flexible sigmoidoscope.” First, a gastroenterologist will expose the patient's anus and gently insert a gloved and lubricated finger into the rectum to check for blockage and dilate the anus. This is called a digital rectal examination. Following the digital rectal exam, the sigmoidoscope will be inserted. This is a flexible tube about 20 inches long. The scope is gently advanced into the colon. Air is introduced into the scope to aid in viewing. The air may cause the urge to defecate. As the sigmoidoscope is slowly removed, the lining of the bowel is carefully examined. A channel in the scope allows for the passage of forceps for biopsies or other instruments for therapy. This test can help diagnose inflammatory bowel disease, bowel obstruction, colon cancer, colon polyps, diverticulosis, causes of diarrhea and causes of abdominal pain. This test can also be used to determine the cause of blood, mucus, or pus in the stool, confirm findings of another test or X-rays and take a biopsy of a growth.

A colonoscopy is a procedure for viewing the interior lining of the large intestine (colon) using a colonoscope (which is a flexible tube containing an imaging device). The patient lies on his or her left side with the knees drawn up toward the abdomen. After administration of an intravenous sedative and analgesic, the instrument is inserted through the anus and gently advanced under direct vision to the terminal small bowel. Air will be inserted through the scope to provide a better view. Suction may be used to remove secretions. Since better views are obtained during withdrawal than during insertion, a more careful examination is done during withdrawal of the scope. Tissue samples may be taken with tiny biopsy forceps inserted through the scope. Polyps can be removed with electrocautery snares, and photographs can be taken. Specialized procedures, such as laser therapy, can also be performed. The test is performed to obtain tissue specimen for biopsy; to evaluate unexplained blood in the stool, abdominal pain, persistent diarrhea, or abnormalities (such as polyps) found on contrast X-rays (barium enema); to determine the type and extent of inflammatory bowel disease (ulcerative colitis and Crohn's disease); and to follow people with previous polyps, colon cancer, or a family history of colon cancer.

Esophagogastroduodenoscopy (EGD) is a test that involves visually examining the lining of the esophagus, stomach, and upper duodenum with an endoscope that is inserted down the throat. The patient will be given a sedative and an analgesic. A local anesthetic will be sprayed into the mouth to suppress the need to cough or gag when the endoscope is inserted. A mouth guard will be inserted to protect the teeth and the endoscope. An IV may be inserted to administer medications during the procedure. The patient is instructed to lie on their left side. After the gag reflex has been suppressed by the anesthetic, the endoscope will be advanced through the esophagus to the stomach and duodenum. Air will be introduced through the endoscope to enhance viewing. The lining of these organs is examined and biopsies can be obtained through the endoscope. When the area has been viewed and any biopsies taken, the endoscope will be removed and the patient will be asked to cough to expel the extra air. Food and liquids are restricted until cough reflex returns. The test lasts about 30 to 60 minutes. This test is helpful in determining: the cause of upper GI (gastrointestinal) bleeding; the cause of swallowing difficulties; the presence of ulcerations or inflammation; the cause of abdominal pain; the condition of the stomach and duodenum after an operation; the presence of tumors or other abnormalities of the upper GI tract; and inflammation, narrowing, or tumors of the esophagus.

ERCP is an X-ray of the pancreatic ducts and biliary tree, which provide enzymes used in digestion. The test is used to look for stones or tumors in the ducts, a narrowing of the ducts, or cancer. The patient's throat is sprayed with a local anesthetic. A sedative and pain killer is given through a vein. A special flexible endoscope is inserted through the mouth into the duodenum. A catheter is advanced through the endoscope and inserted into the pancreatic or biliary ducts. A contrast agent is injected into these ducts and X-rays are taken to evaluate their caliber, length and course. Narrowing, stones, and tumors can be identified. Special instruments can be placed through the scope and into the ducts to open the entry of the ducts into the bowel, stretch out narrow segments, remove or crush stones, take tissue samples, and drain obstructed areas. The procedure identifies any abnormality of the pancreas or bile ducts that can cause abdominal pain, jaundice, fever, or malabsorption. These include gallstones, bile duct strictures, bile duct tumors, chronic pancreatitis, pancreatic tumors (including pancreatic cancer), pancreatic strictures, and pancreatic pseudocysts.

Small bowel biopsy is a diagnostic procedure in which a portion of the lining of the small bowel (small intestine) is removed for examination. Small bowel biopsy samples can be obtained by EGD or other endoscopy of the upper gastrointestinal tract. A flexible tube or endoscope is inserted through the mouth or nose and into the upper gastrointestinal tract. Tissue samples removed during endoscopy are sent to the laboratory for examination.

Capsule biopsy produces a larger sample of the intestinal lining (mucosa) and allows sampling of areas that are beyond the reach of the endoscope. The procedure is similar to that of EGD. The back of the patient's throat is sprayed with a local anesthetic to prevent gagging. The tube and capsule is inserted through the mouth and the patient is asked to swallow as the tube is advanced. The position may be changed from sitting to lying on the right side to help the capsule advance through the stomach and into the small bowel. When the capsule is properly positioned, suction is applied to the tube (which causes the capsule to close and grab tissue). Once a tissue sample has been obtained, the tube and capsule are removed. This test is most often performed to help diagnose diseases of the small intestines.

Arthroscopy is a method of viewing or performing surgery on a joint by use of an arthroscope, which consists of a tube, a lens, and a light source utilizing fiber optics to visualize the surgical area. Typically, this procedure is performed on the knee joint. A local or regional anesthetic is administered, which numbs the affected area, but the patient remains awake and able to respond. For more extensive surgery, general anesthesia may be used. In this case the patient is unconscious and pain-free. The area is cleaned with antiseptic soap. A pressure band (tourniquet) may be applied to restrict blood flow. An incision is made into the joint, and sterile fluid is introduced into the joint space to provide a better view. The arthroscope is then inserted, and the inside of the joint is viewed by displaying the image on a monitor. One or two small additional incisions by the knee may be needed, in order to use other instruments. These instruments can be used to remove bits of cartilage or bone, take a tissue biopsy, or perform other minor surgery. In addition, ligament reconstruction can be performed mostly using the arthroscope in many cases. The procedure is similar for the shoulder except for the band used to restrict blood flow. Also, the patient is usually asleep in shoulder arthroscopy. Diagnostic or simple arthroscopy usually lasts about 1 hour.

Bronchoscopy is a diagnostic procedure in which a tube with a tiny camera on the end is inserted through the nose or mouth into the lungs. The procedure provides a view of the airways of the lung and allows doctors to collect lung secretions and to biopsy for tissue specimens. The pulmonologist (a lung specialist trained to perform a bronchoscopy) sprays a topical or local anesthetic in the patient's mouth and throat. This will cause coughing at first, which will cease as the anesthetic begins to work. When the area feels “thick,” it is sufficiently numb. Medications to relax the patient may be given through an IV. If the bronchoscopy is performed via the nose, an anesthetic jelly will first be inserted into one nostril. When it is numb, the scope will be inserted through the nostril until it passes through the throat into the trachea and bronchi. Usually, a flexible bronchoscope is used. The flexible tube is less than ½-inch wide and about 2-feet long. As the bronchoscope is used to examine the airways of the lungs, the doctor can obtain samples of lung secretions to send for laboratory analysis. Saline solution can be introduced to flush the area and collect cells that may be analyzed by a pathologist or microbiologist. This part of the procedure is called a “lavage” or a bronchial washing. Usually, small amounts (5-10 cc, or 1-2 teaspoons) of saline are used. In certain circumstances, a larger volume of saline may be used. In this procedure, called bronchoalveolar lavage, up to 300 cc of saline (20 tablespoons) are instilled into the airway after the bronchoscope has been advanced as far as possible and a small airway is completely blocked (temporarily) by the scope. Bronchoalveolar lavage is performed to obtain a sample of the cells, fluids, and other materials present in the very small airways and alveoli. In addition, tiny brushes, needles, or forceps can be introduced through the bronchoscope to obtain tissue samples from the lungs. Occasionally, stenting and laser therapies can be performed through the bronchoscope. A rigid bronchoscope is less commonly used, and usually requires general anesthesia. This test is recommended if a chest X-ray or other diagnostic procedure suggests a lung disease that requires an inspection of the airways of the lung or a tissue sample for diagnosis.

Cystoscopy is a procedure that enables the health care provider to view the inside of a patient's bladder and urethra in great detail using a specialized endoscope called a cystoscope. There are two types of cystoscopes, the standard rigid cystoscope and the flexible cystoscope. The method for insertion of the cystoscope varies, but the test is the same. The choice of which scope to use depends on the purpose of the exam. If the standard rigid cystoscope is used, the patient lies in the lithotomy position—on back with the knees up and apart. The flexible cystoscope may be easier to insert than the standard rigid model. It does not require the lithotomy position for insertion. The procedure usually takes between 5 and 20 minutes. The urethra is cleansed and a local anesthetic is applied. The scope is then inserted through the urethra into the bladder. Water or saline is inserted through the cystoscope and fills the bladder. As the fluid fills the bladder, it stretches the bladder wall, enabling the physician to view the entire bladder wall. If any tissue appears abnormal, a small specimen can be taken (biopsy) through the cystoscope to be analyzed. This procedure is used to diagnose and evaluate urinary tract disorders, check for cancer of the bladder or urethra, diagnose an enlarged prostate, help determine the cause of pain during urination, and diagnose recurrent bladder infections.

Diagnostic laparoscopy is a procedure that allows a health care provider to look directly at the contents of a patient's abdomen or pelvis, including the fallopian tubes, ovaries, uterus, small bowel, large bowel, appendix, liver, and gallbladder. The purpose of this examination is to directly assess the presence of a problem that has not been confirmed through noninvasive tests. Inflammation of the gallbladder (cholecystitis), appendix (appendicitis), or pelvic organs (pelvic inflammatory disease) or tumors of the ovaries may be diagnosed laparoscopically. Additionally, the health care provider may wish to exclude abdominal trauma following an accident by using laparoscopy rather than a large abdominal incision. Major procedures to treat cancer, such as surgery to remove an organ, may begin with laparoscopy to exclude the presence of additional tumors (metastatic disease), which would change the course of treatment. The procedure is usually done in the hospital or outpatient surgical center under general anesthesia. However, this procedure may also be done using local anesthesia, which merely numbs the area affected by the surgery and allows the patient to stay awake. A small incision is made below the navel to allow the insertion of a tube called a trocar, which allows passage of a tiny video camera into the abdomen. Prior to insertion of the trocar, a needle is inserted into the incision and carbon dioxide gas is injected to elevate the abdominal wall, thereby creating a larger space to work in. This allows for easier viewing and manipulation of the organs. The laparoscope is then inserted so that the organs of the pelvis and abdomen can be examined. Additional small incisions may be made for instruments that allow the surgeon to move organs for more complete visualization. In the case of gynecologic laparoscopy, dye may be injected through the cervical canal to make the fallopian tubes easier to view. Following the examination, the laparoscope is removed, the incisions are closed, and bandages are applied. The examination helps identify the cause of pain in the abdomen and pelvic area. It may detect the following conditions: endometriosis (tissues normally found in the uterus growing in other areas); ectopic pregnancy (in which the fertilized egg develops outside of the uterus); pelvic inflammatory disease (an inflammation in the pelvic cavity); cancer; cholecystitis; and appendicitis.

With the use of endoscopes with all of these procedures, the commonality is the use of a camera to assist the health care provider in directing the endoscope as well as looking for abnormalities that are to be treated. Endoscopes are designed either with a single camera attached to the distal end of the flexible tube or with a fiberoptic bundle that transmits an image from a lens at the distal end of the scope to an eyepiece or video camera at the proximal end. Accordingly, a scope provides for a two dimensional visual feedback from the prospective of the position of the end of the scope. To capture an image from another angle or an image in a different portion of the body cavity, the endoscope needs to be repositioned or moved back and forth. Repositioning and movement of the scope back and forth can prolong the time of the procedure and provide added discomfort, complications, and risks to the patient. Additionally, a large growth in a lumen can prevent the advancement of the scope until its removal. Under these circumstances, a health care provider only sees the front end of the growth, with little awareness of its depth or what may exist behind it.

### SUMMARY

In accordance with one aspect of the present invention, a catheter assembly is provided comprising a catheter handle coupled to an insertion tube, the insertion tube including at least one lumen extending at least partially through the insertion tube; a first imaging element positioned at or near a distal end of the insertion tube for providing a first visual reference for a health care provider; and a second imaging element configured to be extended out from and retracted into the lumen of the insertion tube, the second imaging element being movable with respect to the first imaging element so as to provide a health care provider a second visual reference or multiple visual references. The catheter assembly can additionally include an imaging tube configured to be inserted into a port of the catheter or catheter handle and slidably disposed in the lumen of the insertion tube, the imaging tube including the second imaging element at or near a distal end or distal tip thereof. In some embodiments, the first and/or second imaging elements can include a lens, a single chip sensor, a multiple chip sensor and the like. The imaging tube can include a bendable or steerable distal segment so as to allow for adequate or optimum imaging. In some embodiments, the bendable or steerable distal segment can be made from a shape memory material, spiral cut hypotube, a spring structure or the like. In some embodiments, a means for bending a distal segment of the imaging tube can be provided. For example, a steering mechanism for directing the movement of a distal segment of the imaging tube can be included. The catheter assembly can include a second lumen disposed through the insertion tube for receiving a therapeutic and/or diagnostic tool.

In some embodiments, the second imaging element includes an elongated tube having a cutout portion at or near a distal end or tip of the elongated tube and an imaging component positioned on a distal side of the cutout portion. The elongated tube can include a bendable or steerable distal segment. In some embodiments, the second imaging element includes a guidewire or guidewire structure, such as the type over which a catheter can be guided.

In some embodiments, the second imaging element includes an elongated tube having a generally V (or “L”) shaped or U shaped cutout portion at or near a distal end or tip of the elongated tube and an imaging component positioned on the distal side of the cutout portion.

In other embodiments, the second imaging element includes an elongated tube having a cutout portion at or near a distal end or tip of the elongated tube; a mirror positioned on a distal side of the cutout portion; and an imaging component positioned on a proximal side of the cutout portion for receiving the image off of the mirror.

In some embodiments, reflectors and lighting elements, such as light emitting diodes (LEDs) are also provided.

In some embodiments, a means for directing the movement of the insertion tube is provided. This can, for example, include a steering mechanism for causing directional movement of a distal end of the insertion tube.

In accordance with another aspect of the invention, a catheter assembly is provided comprising a first catheter device including an insertion tube and a first imaging device coupled to a distal end of the insertion tube; and a second catheter device having an imaging tube, the imaging tube configured to be slidably disposed in the insertion tube and to extend out from the insertion tube, the second catheter device comprising a second imaging device that can be moved with respect to the first imaging element. In some embodiments, the first catheter device includes a steering mechanism for directing the movement of the insertion tube. In some embodiments, the second catheter device includes a steering mechanism for directing the movement of the imaging tube.

In accordance with another aspect of the invention, a catheter assembly is provided comprising a first catheter device including an insertion tube and a first imaging device at or near a distal end or tip of the insertion tube; a second catheter device for receiving the insertion tube of the first catheter device; and an imaging tube capable of being extended through and disposed out from a lumen of the second catheter device, the imaging tube having a second imaging device at or near a distal end or tip thereof.

In accordance with another aspect of the invention, a method for diagnosing and/or treating a lesion is provided by using any of the above described medical assemblies. In some embodiments, for example, the method can include positioning a first catheter assembly in front of a designated lesion; capturing a first image of the lesion with a first visual device integrated near or at a distal end or tip of the first catheter assembly; extending a second catheter assembly (or guidewire assembly, imaging lumen, or the like) through the first catheter assembly and past the first visual device; and capturing a second image of the lesion with a second visual device integrated near or at a distal end or tip of the of the second catheter assembly (or guidewire assembly, imaging lumen or the like). The first and second images can be live video feedbacks. The second visual device can be positioned behind the lesion so as to provide both front and back images. The second visual device can traverse (back and forth) the side or top of the lesion so as to provide more information to the health care provider about the lesion. The method can additionally include performing a therapeutic or diagnostic procedure contemporaneously with capturing the first and second images or subsequent to capturing of the images.

### DESCRIPTION OF THE FIGURES

FIG. 1 is one embodiment of the catheter assembly of the present invention;

FIG. 2 is a side view of a distal section of FIG. 1;

FIG. 3 is a perspective view of a distal section of FIG. 1;

FIGS. 4A, 4B, 4C, 4D1, 4D2, 4E, and 4F are various embodiments of an imaging or visual device at a distal section of the catheter assembly;

FIGS. 5, 6, and 7 are components of the catheter assembly in accordance with another embodiment of the present invention; and

FIG. 8 is schematic illustration of an imaging device in accordance with another embodiment of the invention.

### DETAIL DESCRIPTION OF THE EMBODIMENTS OF THE INVENTION

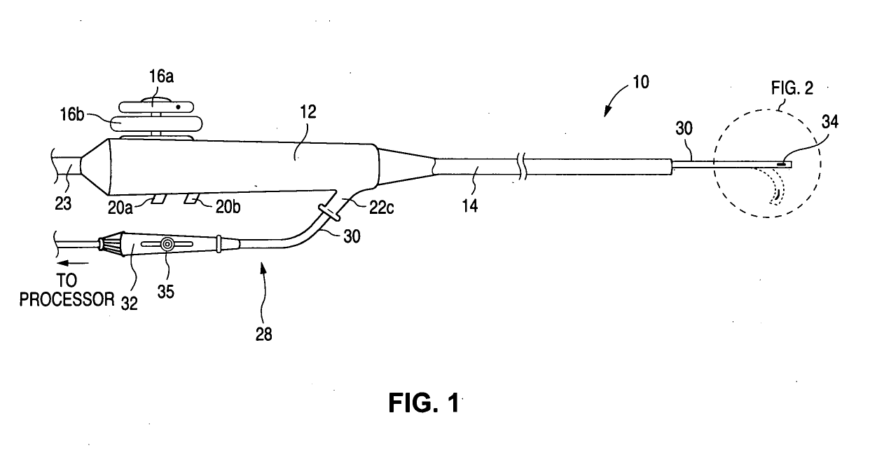

Referring to FIGS. 1, 2, and 3, there is illustrated a catheter assembly 10 in accordance to one embodiment of the invention. This catheter assembly 10 can be used in any variety of medical procedures in which imaging of a body tissue, organ, cavity or lumen is required. The types of procedure include those discussed in the background section, including, for example, anoscopy, arthroscopy, bronchoscopy, colonoscopy, cystoscopy, EGD, laparoscopy, and sigmoidoscopy. The catheter assembly 10 includes a control handle 12 to which an insertion tube 14 is connected. The insertion tube 14 can be detachable from the control handle 12 or can be an extension or in permanent connection. The diameter, length and flexibility of the insertion tube 14 depend on the procedure for which the catheter assembly 10 is used. The insertion tube 14 can be made from or coated with a lubricious material so as to allow for easy insertion and extraction of the insertion tube from a patient. Catheter materials and constructs are well known in the art.

The control handle 12 can include control knobs 16a and 16b (16) that are attached to four control cables or wires 18 (FIG. 3) for the manipulation of the insertion tube 14. In some embodiments, a clutch or breaking component could be included with the control knobs 16 as to preventing the knobs 16 from inadvertently rotating such that rotation can only be caused by application of a certain degree of torque to the knobs 16. The control cables 18 are symmetrically positioned within a core of the insertion tube 14 and extend along the length of the insertion tube 14. The control cables 18 are anchored at or near the distal end of the insertion tube 14 such that the rotation of the control knobs 16a and 16b in a clockwise or counter clockwise direction accounts for up and down as well as side to side movement for the insertion tube 14. The operation of a combination of two adjacent wires 18 can provide for angular movement of the insertion tube 14. This type of control mechanism is well known by one having ordinary skill in the art. It should be noted that a single control knob can also be used with one or two wires.

Control handle 12 includes multiple ports and/or valves, two of which are illustrated by way of example by reference numbers 20a and 20b (20). The ports and/or valves 20a and 20b are in communication with their respective lumens 22a and 22b (FIG. 3) extending through the insertion tube 14. Although two ports and valves 20 are illustrated by FIG. 1, any number can be used. The only compromise with the use of a multitude of ports and valves 20 is that each can correspond with a lumen that extends through the insertion tube 14. As a result, size considerations must be taken into account as additional catheter lumens may comprise the functionality of the device. “Y” junctions can be used to designate two ports to a single lumen or one port to two lumens. The ports and or valves 20a and 20b can be air/water valves, suction valve, instrumentation port as well as suction/instrumentation port. In some embodiments, one of the lumens can be used for a wash channel in that pressurized water can be ejected out from an opening at a distal end of lumen 22. A cap (not shown) can be included at the opening to divert the water onto a lens of an imaging component for cleaning. Others channels can be used to apply a gas, such as CO₂ into the organ. Lumens can also be used to extract fluids or inject fluids, such as water or a drug in a liquid carrier, into the body. Various biopsy, drug delivery, balloon catheters and other devices which can be diagnostic and/or therapeutic in nature can also be inserted via the lumens to perform specific functions. In some embodiments, various tools include a retractable needle for drug injection, hydraulically actuated scissors, clamps, grasping tools, electrocoagulation systems, ultrasound transducers, electrical sensors, heating elements, laser mechanisms and other ablation means.

An accessory outlet 23 at a proximal end of the control handle 12 allows for air, water and suction channels to be in fluid communication with pumps and related accessories. The same outlet or a different outlet can be used for electrical lines to light and imaging components at a distal end of the insertion tube 14.

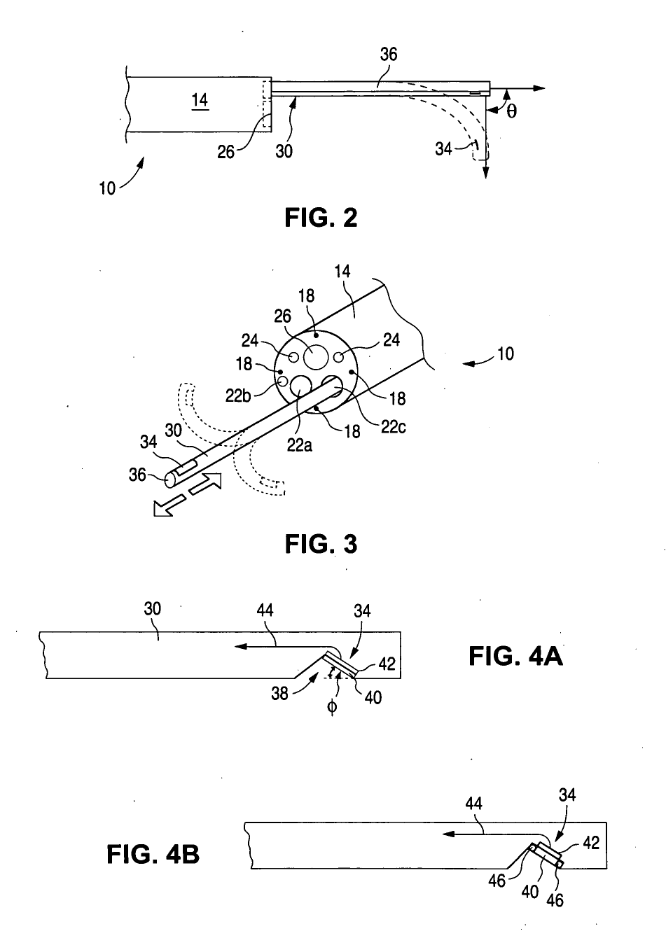

As illustrated by FIGS. 2 and 3, the insertion tube 14 additionally includes lighting elements such as light emitting diodes (LEDs) 24 and an imaging or visual device 26. The visual device 26 can be at a fixed position or coupled to an end of the insertion tube 14 and can include a lens, single chip sensor, multiple chip sensor or fiber optic implemented devices, for example. “Fixed” is defined as not being able to move relative to the insertion tube 14. The visual or imaging device 26, in electrical communication with a processor and/or monitor, can be for taking single or still images or recorded or live video images. Control knobs (not shown) can be included in the control handle 12 for controlling image functions such as focus, brightness, sharpness, etc. as well as the intensity of the LEDs 24. Each LED 24, individually, can be turned on or off. The intensity of each can also be adjusted so as to achieve optimum imaging.

As best illustrated by FIGS. 1, 2, and 3 the catheter assembly 10 additionally includes a movable imaging catheter assembly 28 that includes a movable imaging tube 30 coupled to a control handle 32. In some embodiments the imaging tube 30 has a diameter of less than 4 mm. The imaging tube 30 can be inserted into port 22c and extended through a lumen 22c of the insertion tube 14. The imaging tube 30 is slidably disposed within the insertion tube 14 so as to allow a visual or imaging device 34 of the imaging catheter assembly 28 to be movable with respect to the imaging or visual device 26 of the insertion tube 14. In some embodiments, the imaging or visual device 34 should be very compact such has having a size (e.g., in cross section) less than 4 mm. In one embodiment, adequate number of lumens is provided so as to allow for a therapeutic and/or diagnostic tool to be used simultaneously with the imaging tube 30. Accordingly, multidirectional views can be captured before, during and/or after the performance of a diagnostic or therapeutic function, such as tissue ablation or polyp removal. As illustrated by FIG. 1, the control handle 32 can include a steering mechanism 35. In one embodiment, the steering mechanism 35 can be single control knob or a double control knob. A single control knob can be connected to a single wire 36 or two wires for up/down or side movement of a distal segment of the imaging tube 30 (see FIG. 2). A double control knob can provide for both up and down and side to side movement as well as angular movement should both knobs be used in concert. Preferably a single wire will be sufficient as the rotation of the imaging tube 30 within the insertion tube 14 can compensate for the lack of additional wires.

The distal segment of the imaging tube 30 needs to be flexible enough so as to allow for bending or steering of the device. The imaging tube 30 can have a variable stiffness across the length of the tube so that one on hand the distal segment is flexible enough so as to allow for adequate viewing angles and on the other hand the remaining portion stiff enough so as to allow maneuverability of the imaging tube 30 within the insertion tube 14. Maneuverability includes back and forth movement and/or rotational movement of the imaging tube 30 within the lumen 22c.

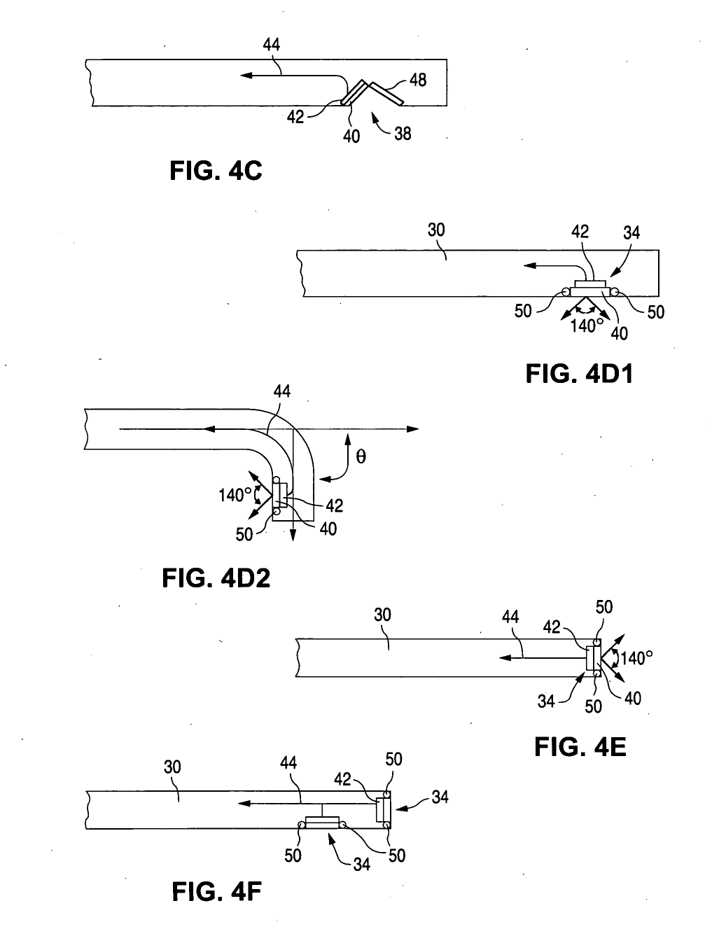

In some embodiments, the imaging tube 30 can be generally unbending or stiff across the entire length of the tube 30. The imaging tube 30 should, however, be flexible enough so as to perform its intended function of being contoured around bends or curves of a bodily lumen. In some embodiments, the imaging tube 30 can include an extremely flexible distal end length so as to allow for bending of the distal end length in response to the steering mechanism 35. Accordingly, a distal length can be bowed, curved, flexed or bent to an angle θ (FIGS. 2 and 4D2) of at least 90 degrees with respect to the remaining length of the tube 30. In some embodiments, the angle θ can be between 0 degrees and 45 degrees. In some embodiments, the angle can be between 0 degrees and less than 95 degrees. As a result, the visual or imaging device 34 can be wrapped around a lesion or directed to a specific or desirable direction.

FIG. 4A illustrates one embodiment of the distal end of the imaging tube 30. A cutout 38 is at a position in close proximity to or at an end of imaging tube 30. In one embodiment, the cutout 38 can be “V” shaped. Unless otherwise specified, “V” shaped is intended to include “L” shaped or in other words when the V angle is greater and one length is longer than the other. In some embodiments, the cut out can be “U” shaped or in other words have a curved transition without any hard corners or angles. On a distal end of the cutout 38 a component of the visual or imaging device 34 is located. The component can include a lens 40 and single chip or multiple chip sensor 42. As best illustrated by the figure, the lens 40 is titled at an angle φ of preferably 45 degrees or approximately 45 degrees. The angle (p can, in some embodiments, be between 20 degrees and 70 degrees, 30 degrees and 60 degrees, 40 degrees and 50 degrees. An electrical wire 44 (wire can be a single wire or a bundle of wires, collectively referred to as “a wire”) connects the imaging component to a processor and/or monitor for viewing, capturing, recording and/or saving of the images. With respect to FIG. 4A, in one embodiment, a distal length of the imaging tube 30 need not be bendable, although it could be, as the imaging component 34 is configured or angled to capture an image away from or behind the distal end of the imaging tube 30. In the bendable configuration, a distal length of the imaging tube 30 can be made from a shape memory plastic that is preformed in a bent, bowed, curved, or flexed configuration. Shape memory can also be provided by metal or spring steel components as is known to one having ordinary skill in the art. As a result, when the distal length of the imaging tube 30 is pushed out from the distal end of the insertion tube 14, it bends to its designated shape. Rotation of the imaging tube 30 in the insertion tube 14 can provide directional controllability to a user.

FIG. 4B illustrates another embodiment which is the same as FIG. 4A but for the inclusion of reflector plates 46. Light from the LEDs 24 of the insertion tube 14 (FIG. 3) can bounce off the reflectors 46 for providing adequate lighting for the imaging device 34. In lieu of reflectors, LEDs can also be positioned in close proximity of the lens 40. Preferable only one LED should be sufficient but the addition of a second LED should account for ample lighting. Control handle 32 can include a mechanism for turning the LEDs on or off or for adjusting the intensity of the LEDs. Control over the operation of the LEDs can also be made by a processor that can be in electrical communication with the LEDs. With the use of two or more LEDs, a user should also have the option of being capable of independently controlling each one, such as turning one off or adjusting the intensity so as to enhance the image or to obtain the optimum image. FIG. 4C is another embodiment of the invention. In the distal end or side of the cutout 38 (e.g., V shaped), a mirror 48 is fixed. The lens 40 and sensor 42 are positioned on the proximal, opposing end or side of the cutout 38 so as to capture the image off of the reflection in the mirror 48. As before, reflectors or LEDs can be strategically positioned so as to provide for adequate lighting. Moreover, a distal length of tube 30 can be bendable via a control wire 36 or a distal length can be made from a shape memory material, as both options are applicable to all embodiments of the invention.

FIGS. 4D1 and 4D2 illustrate another embodiment of the invention. In lieu of having a cut out 38 with an angle (p so as to provide for a field of vision in a backwards direction, the imaging device 34 is positioned on a side of and at a distance from an end tip of the imaging tube 30. As discussed before, a steering means can be used to bend or flex a distal length of the imaging tube 30 at an angle θ. In some embodiments, a shape memory material can be used at a distal length of the tube 30 so as to allow for bending of a distal section of the imaging tube 30. The imaging device 34 can have a field of vision of about 140 degrees so as to enable adequate visual feed back to a health care provider. The lens 40 and sensor 42 can be between two LEDs 50.

In FIG. 4E the lens 40 is positioned at the distal tip of the imaging tube 30 as opposed to a position inward from the distal tip as shown in FIG. 4D. LEDs 50 provide the lighting. With the bending of the distal length of the tube 30 and a 140 degree field of vision, adequate visual coverage should be provided. Referring to FIG. 4F, two visual devices 34 can be provided, which is in essence the combination of FIGS. 4D and 4E.

The embodiments of the present invention provide for two visual or imaging elements, one at a fixed position at the distal end of the insertion tube 14 and another at a movable position through a lumen of the insertion tube 14. A health care provider can inspect a lesion such as a cancer or polyp at various angles. The movable visual means can be positioned, for example, at a side or past the lesion so as to provide a back angle of the lesion. The added images can advantageously provide the heath care provider with more information with respect to the site in need of treatment. In some embodiments, a third port and lumen of insertion tube 14 can be provided to allow for a diagnostic and/or therapeutic tool, such as a biopsy tool, to be used as both visual aids are in use. A more accurate extraction of tissue can be accomplished this way.

A second auxiliary camera will dramatically improve the ability of physicians to detect cancers and other abnormalities that could be missed when using conventional endoscopes like a colonoscope. It will also reduce the risk of perforation of the bowel during removal of polyps and other abnormal lesions. The human colon has many twists and turns, as well as innumerable folds in its walls. The likelihood of an abnormal lesion in the colon not being detected is highly correlated with its location. Lesions that are just beyond a curve, or hidden behind a fold in the wall, are much more likely to be missed. The auxiliary camera provides a second point-of-view to reveal hidden polyps. The auxiliary camera can project beyond the tip of a catheter and can be aimed backward, toward the tip of the instrument. As the scope is withdrawn during colonoscopy, the cameras provide views of the opposite side of each fold or sharp curve—and of any cancers, polyps or other lesion that might be hidden. The auxiliary camera also offers an additional margin of safety during biopsies and polypectomies. One of the greatest causes for bowel perforation during colonoscopies is the tendency to cut too deeply during polypectomy. Polyps are usually removed by inserting a wire loop through an instrument channel and placing it around the base of the polyp. The loop is then gradually tightened while an electric current through the loop cauterizes the base to prevent bleeding. Unfortunately, it is generally not possible to observe the exact position of the loop, since it is hidden behind the polyp. If the loop is placed too far from the wall, part of the abnormal lesion will be left behind. If the loop is too close to the wall, the cautery can burn through the wall, resulting in a perforation. This is a serious complication, and if it is not immediately recognized, it can result in the death of the patient. However, if the auxiliary camera has been inserted through a second instrument channel, it can provide a view of the opposing side of both the polyp and the wire loop, allowing more precise placement of the loop and reducing the risk of perforation. The auxiliary camera can be inexpensive and can be made fully disposable.

While particular embodiments of the present invention have been shown and described, it will be obvious to those skilled in the art that changes and modifications can be made without departing from this invention in its broader aspects. For example, should the catheter assembly 10 not be fully or partially disposable, the catheter assembly 10 can include a disposable sheath or a protective catheter in which the insertion tube 14 can be disposed. The disposable sheath or protective catheter will protect at least the insertion tube 14, and optionally the control handle 12, from being exposed to bodily fluid, tissues and contaminants. The distal tip of the disposable sheath or protective catheter can include a window so as to allow imaging device 26 of the insertion tube 14 to capture images. The major difference with FIG. 1 would be that the disposable sheath or protective catheter will include a lumen in which the imaging tube 30 can be disposed and extended out from the distal end of the instrument. The disposable sheath or protective catheter can also include other lumens for diagnostic and/or therapeutic tools as well as for application or extraction of fluids and gases.

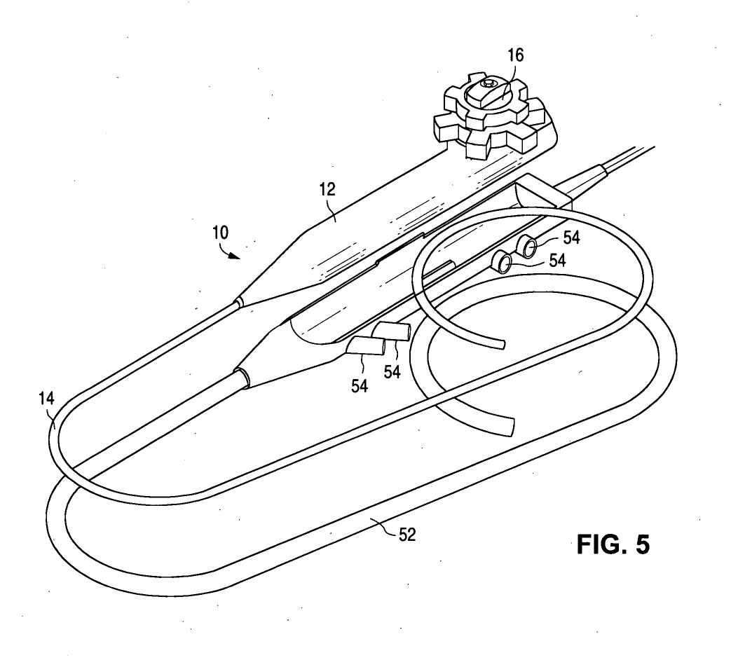

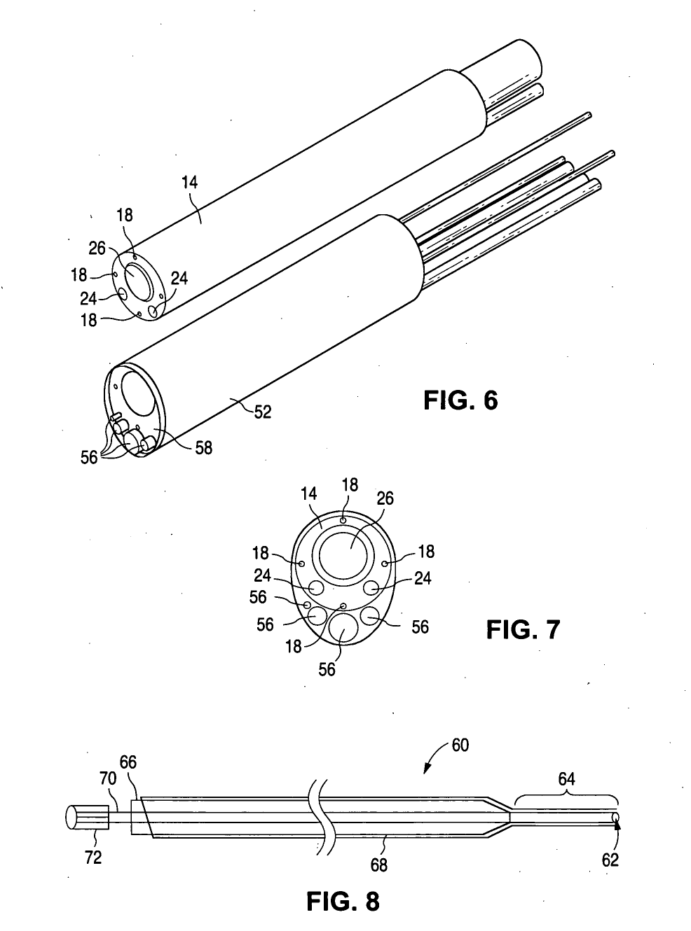

Referring to FIG. 5, the catheter assembly 10 is illustrated having the control handle 12 and insertion tube 14. The control mechanism 16 can be located on the proximal end of the control handle 12. A second catheter assembly 52 can be used as a disposable catheter to protect the insertion tube 14 from exposure to bodily fluids, tissues and contaminants. The insertion tube 14 is extended through a lumen of the second catheter assembly 52. The second catheter assembly 52 includes ports/and valves 54 in communication with lumens 56 extending through the second catheter assembly 52 for air, gas, suction, diagnostic or therapeutic tools as well as for the movable imaging catheter assembly 28. Referring to FIGS. 6 and 7, again, the insertion tube 14 can include the visual or imaging device 26, light sources (LEDs) 26 and control wires 18. The second catheter assembly 52 includes a transparent window 58 end capping the lumen in which the insertion tube 14 is disposed so as to allow for images to be captured while protecting the insertion tube 14 from making contact with fluids and tissues.

In some embodiments movable imaging catheter assembly 28 can be in the form of a guidewire or elongated tube or conduit. In this embodiment, a handle need not be included. The guidewire can be of the type that allows a catheter to be guided through a patient. Referring to FIG. 8, a guidewire 60 is illustrated to include a visual or imaging device 62 (optionally with a light source). A distal segment 64 of the guidewire 60 can be reshaped or preshaped, include a shape memory alloy, be made from NiTi hypotube with a spiral cut, or be made from NiTi or stainless steel coil. The guidewire 60 can include a core wire 66. The core wire 66 can be polymer coated or include a polymeric sleeve 68. Such coating or sleeve 68 can be made from extrusion, solvent spray or dip application, vapor or plasma deposition, or other processes known to one having ordinary skill in the art. In some embodiments, the polymer sheath can be shrink wrapped around the core wire 66 of the guidewire 60. A conductor 70 can traverse the guidewire 60 for allowing visual or imaging device 62 to be in communication with a connector 72 for imaging.

The claims, therefore, are to encompass within their scope all such changes and modifications as fall within the true spirit and scope of this invention.

What is claimed is:

1. A catheter assembly, comprising:
a catheter handle coupled to an insertion tube, the insertion tube including at least one lumen extending at least partially through the insertion tube;
a first imaging element positioned at a distal end of the insertion tube for providing a first visual reference for a health care provider; and
a second imaging element configured to be extended out from and retracted into the lumen of the insertion tube, the second imaging element being movable with respect to the first imaging element so as to provide a health care provider a second visual reference or multiple visual references.

2. The catheter assembly of claim 1, additionally including an imaging tube configured to be inserted into a port of the catheter and slidably disposed in the lumen of the insertion tube, the imaging tube including the second imaging element at a distal end thereof.

3. The catheter assembly of claim 2, wherein the first and/or second imaging element(s) include(s) a lens and a chip sensor.

4. The catheter assembly of claim 2, additionally including a lighting element at the distal end of the imaging tube.

5. The catheter assembly of claim 2, wherein the imaging tube includes a bendable or steerable distal segment.

6. The catheter assembly of claim 5, wherein the bendable distal segment is made from a shape memory material.

7. The catheter assembly of claim 2, additionally comprising means for bending a distal segment of the imaging tube.

8. The catheter assembly of claim 2, additionally comprising a steering mechanism for directing the movement of a distal segment of the imaging tube.

9. The catheter assembly of claim 1, additionally including a second lumen disposed at least partially through the insertion tube for receiving a therapeutic and/or diagnostic tool.

10. The catheter assembly of claim 1, wherein the second imaging element includes
an elongated tube having a cutout portion at or near a distal tip of the elongated tube; and
an imaging component positioned on a distal side of the cutout portion.

11. The catheter assembly of claim 10, wherein the elongated tube includes a bendable or steerable distal segment.

12. The catheter assembly of claim 1, wherein the second imaging element includes
an elongated tube having a generally V or U shaped cutout portion at or near a distal tip of the elongated tube; and
an imaging component positioned on the distal side of the V or U shaped cutout portion.

13. The catheter assembly of claim 12, wherein the elongated tube includes a bendable or steerable distal segment.

14. The catheter assembly of claim 1, wherein the second imaging element includes
an elongated tube having a cutout portion at or near a distal tip of the elongated tube;
a mirror positioned on a distal side of the cutout portion; and
an imaging component positioned on a proximal side of the cutout portion for receiving the image off of the mirror.

15. The catheter assembly of claim 14, wherein the elongated tube includes a bendable or steerable distal segment.

16. The catheter assembly of claim 1, additionally including a lighting element positioned next to the first imaging element and reflectors positioned next to the second imaging element for reflecting the light from the lighting element.

17. The catheter assembly of claim 1, additionally including means for directing the movement of the insertion tube.

18. The catheter assembly of claim 1, additionally including a steering mechanism for causing directional movement of a distal end of the insertion tube.

19. The catheter assembly of claim 1, additionally including a guidewire configured to be inserted into a port of the catheter and slidably disposed in the lumen of the insertion tube, the guidewire including the second imaging element at a distal end thereof.

20. A catheter assembly comprising:
a first catheter device including an insertion tube and a first imaging device coupled to a distal end of the insertion tube; and
a second catheter device including an imaging tube, the imaging tube configured to be slidably disposed in the insertion tube and to extend out from the insertion tube, the second catheter device comprising a second imaging element that can be moved with respect to the first imaging element.

21. The catheter assembly of claim 20, wherein the first catheter device additionally includes a steering mechanism for directing the movement of the insertion tube.

22. The catheter assembly of claim 20, wherein the second catheter device additionally includes a steering mechanism for directing the movement of the imaging tube.

23. A catheter assembly, comprising
a first catheter device including an insertion tube and a first imaging device at a distal end of the insertion tube;
a second catheter device for receiving the insertion tube of the first catheter device; and
a third catheter device having an imaging tube capable of being extended through and disposed out from a lumen of the second catheter device, the imaging tube having a second imaging device at or near a distal tip thereof.

24. A method for diagnosing or treating a lesion, comprising
positioning a first catheter assembly in front of a designated lesion;
capturing a first image of the lesion with a first visual device integrated at a distal end of the first catheter assembly;
extending a second catheter assembly through the first catheter assembly and past the first visual device;
capturing a second image of the lesion with a second visual device integrated near or at a distal end of the second catheter assembly.

25. The method of claim 24, wherein the first and second images are live video feedbacks.

26. The method of claim 24, wherein the second visual device is positioned behind the lesion.

27. The method of claim 24, additionally comprising performing a therapeutic or diagnostic procedure contemporaneously with capturing the first and second images.

28. The method of claim 27, additionally including moving the second imaging device contemporaneously with the therapeutic or diagnostic procedure.

29. The catheter assembly of claim 1, wherein the second imaging element is smaller than 4 mm in cross section.
